(19) United States
(12) Patent Application Publication
(10) Pub. No.: US 2026/0263826 A1
(43) Pub. Date: Sep. 10, 2026

(54) APPARATUS FOR NON-INVASIVE BRAIN INTERVENTION WITH RADIO FREQUENCY MICROWAVES

(71) Applicant: Xi'an Jiaotong University, Xi'an (CN)

(72) Inventors: Jue WANG, Xi'an (CN); Sinan LI, Xi'an (CN); Jiawen CHEN, Xi'an (CN); Zigang HUANG, Xi'an (CN); Tian LIU, Xi'an (CN); Long LI, Xi'an (CN); Linyan WU, Xi'an (CN); Liming FAN, Xi'an (CN); Badong CHEN, Xi'an (CN); Liangjun CHEN, Xi'an (CN); Hao WU, Xi'an (CN)

(73) Assignee: Xi'an Jiaotong University, Xi'an (CN)

(21) Appl. No.: 19/663,118

(22) Filed: Apr. 29, 2026

Related U.S. Application Data
(63) Continuation of application No. PCT/CN2024/073877, filed on Jan. 24, 2024.

(30) Foreign Application Priority Data
Nov. 10, 2023 (CN) 202311496743.8

(51) Int. Cl. A61N 5/02; H03F 3/24

(52) U.S. Cl. CPC A61N 5/022; H03F 3/245; H03F 2200/451

(57) ABSTRACT

An apparatus for non-invasive brain intervention with radio frequency microwaves includes a light emitting diode (LED) display screen configured to select a to-be-generated initial waveform and set time; a microcontroller unit configured to generate the initial waveform; a chip configured to generate and send an intervention signal according to the initial waveform, processed human bioconductance data, and temperature data; an adjustable power amplifier, where one end of the adjustable power amplifier is connected to the other end of the chip, and the adjustable power amplifier is configured to receive the intervention signal and amplify the intervention signal; and a thin-film lens microstrip line, where the thin-film lens microstrip line is connected to the other end of the adjustable power amplifier and configured to radiate the amplified intervention signal to a human brain, to complete one intervention stimulation on the human brain.

### CROSS-REFERENCE TO RELATED APPLICATIONS

This application is a continuation of International Application No. PCT/CN2024/073877, filed on Jan. 24, 2024, which claims priority to Chinese Patent Application No. 202311496743.8, filed on Nov. 10, 2023. The disclosures of the above-mentioned applications are hereby incorporated by reference in their entireties.

### TECHNICAL FIELD

Embodiments of the present disclosure relate to the technical field of non-invasive brain intervention, and particularly relate to an apparatus for non-invasive brain intervention with radio frequency microwaves.

### BACKGROUND

In the medical field, diagnosis and treatment of brain diseases have long been a research hotspot. Treatment methods for brain diseases include drug therapy, surgical treatment, psychological therapy, rehabilitation therapy, non-invasive radio frequency microwave therapy, and the like. Among those methods, the non-invasive radio frequency microwave therapy has attracted increasing attention from experts and scholars due to its advantages such as non-invasiveness, high efficiency, safety, and simple operation.

The non-invasive radio frequency microwave therapy is a non-invasive brain intervention therapy, and acts on brain nerve cells by penetrating deep brain tissue through electric and magnetic fields of microwave radio frequency, and treats brain diseases by utilizing a non-thermal effect, thus achieving favorable therapeutic effects on the brain diseases.

Conventional non-invasive brain treatment apparatuses have some defects, which are mainly as follows.

Firstly, focusing of a stimulation zone is inaccurate. Conventional brain intervention stimulation apparatuses, such as transcranial magnetic stimulation (TMS), transcranial alternating current stimulation (tACS), and transcranial direct current stimulation (tDCS), require placement of electrodes on specific scalp zones. However, due to energy dispersion, individual differences, and brain structure complexity, it is difficult to accurately locate the stimulation zone, thus affecting a therapeutic effect.

Secondly, safety is unstable. Owing to complexity of brain nerve cells and varying individual tolerance, existing brain stimulators may cause patient discomforts such as pricking sensation and noise, causing difficulty to achieve painless treatment. Taking the tDCS and the tACS as examples, electrodes attached to a scalp may cause obvious skin stinging or allergic reactions at electrode attachment positions of a patient during stimulation. In view of this, a safety threshold should be strictly limited during stimulation.

Lastly, intervention energy is high. Conventional brain stimulation apparatuses deliver high energy. For example, high-voltage large capacitors are charged through the transcranial magnetic stimulation (TMS), generating thousands of amperes of currents flowing through a stimulation coil within 1 millisecond, with instantaneous power reaching a megawatt level. A mode with high voltage and low current is generally used in electrical stimulation, achieving still high overall power.

### SUMMARY

In view of that, in the embodiments of the present disclosure, there is provided an apparatus for non-invasive brain intervention with radio frequency microwaves, to at least partially solve the above problems.

According to a first aspect, in the embodiments of the present disclosure, an apparatus for non-invasive brain intervention with radio frequency microwaves is provided, including a light emitting diode (LED) display screen, where the LED display screen is configured to select a to-be-generated initial waveform and set time; an STM32 microcontroller unit, where one end of the STM32 microcontroller unit is connected to the LED display screen, and the STM32 microcontroller unit is configured to generate the initial waveform; an LMX2594 chip, where one end of the LMX2594 chip is connected to the other end of the STM32 microcontroller unit, and the LMX2594 chip is configured to generate and send an intervention signal according to the initial waveform, processed human bioconductance data, and temperature data; an adjustable power amplifier, where one end of the adjustable power amplifier is connected to the other end of the LMX2594 chip, and the adjustable power amplifier is configured to receive the intervention signal and amplify the intervention signal; and a thin-film lens microstrip line, where the thin-film lens microstrip line is connected to the other end of the adjustable power amplifier and configured to radiate the amplified intervention signal to a human brain, to complete one intervention stimulation on the human brain.

In one implementation, the apparatus further includes a pair of impedance electrodes; and an ADPD700 impedance analysis chip. One end of the ADPD700 impedance analysis chip is connected to the pair of impedance electrodes, and the other end of the ADPD700 impedance analysis chip is connected to the STM32 microcontroller unit. The ADPD700 impedance analysis chip is configured to collect and process the human bioconductance data through the pair of impedance electrodes, and send the processed human bioconductance data to the STM32 microcontroller unit. The human bioconductance data includes an impedance, a conductivity, and a permittivity of a human body.

In another implementation, the apparatus further includes a temperature probe; and an M601 body temperature monitoring chip. One end of the M601 body temperature monitoring chip is connected to the temperature probe, and the other end of the M601 body temperature monitoring chip is connected to the STM32 microcontroller unit. The M601 body temperature monitoring chip is configured to monitor changes in surface temperature of a scalp through the temperature probe, generate the temperature data, and send the temperature data to the STM32 microcontroller unit.

In another implementation, the thin-film lens microstrip line includes a thin-film lens and a microstrip line. The thin-film lens is configured to refract the intervention signal, causing the intervention signal to be focused on the microstrip line, to implement intervention stimulation on a specific zone of the human brain.

The solution of the present disclosure has the following beneficial effects.

Firstly, conventional brain intervention has invasiveness and high energy, leading to a series of risks and complications. The present disclosure is a non-invasive intervention apparatus. The apparatus reduces risks of existing brain intervention methods and decreases an incidence rate of complications and infections in invasive intervention.

Secondly, microwave brain intervention stimulation of the present disclosure may accurately locate a specific zone of a brain for treatment or research. Focused stimulation on brain zones may be implemented through an electrode design of the thin-film lens and the microstrip line.

Thirdly, tests show that the present disclosure may achieve activation of relevant brain zones through power of 10 dBm. Meanwhile, with reference to a specific absorption rate, brain intervention using the solution of the present disclosure does not cause thermal effect damage. Thus, the solution of the present disclosure is lower in energy consumption and higher in safety.

Lastly, the present disclosure uses a printed gold-coated antenna. The apparatus is smaller in overall size, and has better biocompatibility, wide application scenes, and suitability for long-term wearing.

### BRIEF DESCRIPTION OF THE DRAWINGS

In order to more clearly illustrate technical solutions in embodiments of the present disclosure or in the prior art, accompanying drawings required for the description of the embodiments or the prior art will be briefly introduced below. Obviously, the accompanying drawings in the following description are only some embodiments described in the embodiments of the present disclosure, and those of ordinary skill in the art would also be able to derive other drawings from these drawings.

FIG. 1 is a structural diagram of a system of an apparatus for non-invasive brain intervention with radio frequency microwaves according to one embodiment of the present disclosure.

FIG. 2 is a schematic diagram of a front end structure of a thin-film lens microstrip line according to another embodiment of the present disclosure.

FIG. 3 is a schematic structural diagram and a real picture of a microstrip line according to yet another embodiment of the present disclosure.

FIG. 4 is a schematic diagram of a microwave intervention test on a real skull according to yet another embodiment of the present disclosure.

FIG. 5 is a schematic diagram of simulation results of notch characteristics of a microstrip line according to yet another embodiment of the present disclosure.

FIG. 6 is a diagram of simulation results of a microwave computer according to yet another embodiment of the present disclosure.

FIG. 7 is a diagram of expression results of C-fos protein of a rat subjected to microwave intervention according to still another embodiment of the present disclosure.

### DETAILED DESCRIPTIONS OF THE EMBODIMENTS

In order to understand technical features, objectives and effects of embodiments of the present disclosure more clearly, specific implementations of the embodiments of the present disclosure are described with reference to accompanying drawings.

“For example” herein means “serving as an example, instance, or illustration”, and any illustration or implementation described as “for example” herein should not be construed as a more preferred or advantageous technical solution.

To keep figures concise, only parts related to the present disclosure are schematically shown in each figure, and do not represent an actual structure of the present disclosure as a product. In addition, for conciseness and ease of understanding of the figures, in some figures, only one or more of components having an identical structure or function are schematically drawn or labeled.

In order to enable those skilled in the art to better understand the technical solution of the embodiments of the present disclosure, the technical solution in the embodiments of the present disclosure will be clearly and completely described in conjunction with the accompanying drawings in the embodiments of the present disclosure. Obviously, the embodiments described are merely some embodiments rather than all embodiments of the present disclosure. Based on the embodiments of the present disclosure, all other embodiments obtained by those of ordinary skill in the art should fall within the protection scope of the embodiments of the present disclosure.

For ease of understanding, before specific embodiments of the present disclosure are described in detail, the prior art related to an apparatus for invasive brain intervention with radio frequency microwaves is illustratively described first.

Neuromodulation is an important method in the study of brain functions and brain cognition, and is a response of a brain to a specific stimulus. In the prior art, common methods for evoking brain electrical activities mainly include electrical stimulation, optical stimulation, magnetic stimulation, acoustic stimulation, and other methods. An electrical stimulation method achieves accurate localization and requires implantation of electrodes. An optical stimulation method is commonly used to generate visual evoked potentials and perform deep brain stimulation under craniotomy. A magnetic stimulation method may locate brain zones in vitro, and has restricted spatial resolution and penetration depth. An acoustic stimulation method is generally used to generate auditory evoked potentials. Brain tissue is highly sensitive to trauma, and the situation significantly affects brain functions. Thus, existing invasive methods greatly interfere with original weak electrophysiological information of a brain, and cannot accurately and effectively extract signal features for dynamic research on functional information features.

In view of this, the present disclosure provides an apparatus for non-invasive brain intervention with radio frequency microwaves. Specific implementation of the embodiments of the present disclosure will be further described below in conjunction with the accompanying drawings of the embodiments of the present disclosure.

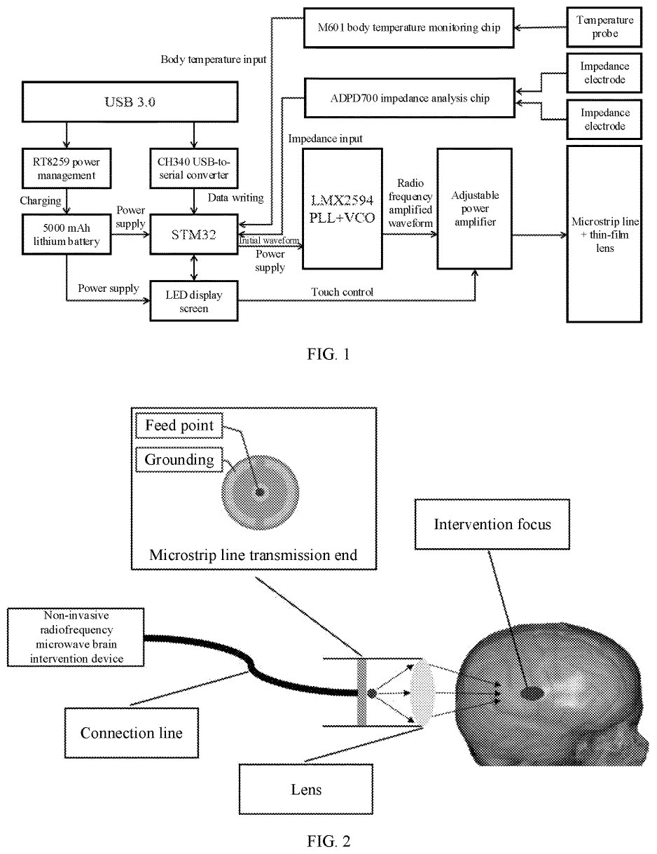

FIG. 1 is a structural diagram of a system of an apparatus for non-invasive brain intervention with radio frequency microwaves according to one embodiment of the present disclosure. The system mainly includes:
a light emitting diode (LED) display screen, where the LED display screen is configured to select a to-be-generated initial waveform and set time;
an STM32 microcontroller unit, where one end of the STM32 microcontroller unit is connected to the LED display screen, and the STM32 microcontroller unit is configured to generate the initial waveform;
an LMX2594 chip, where one end of the LMX2594 chip is connected to the other end of the STM32 microcontroller unit, and the LMX2594 chip is configured to generate and send an intervention signal according to the initial waveform, processed human bioconductance data, and temperature data;
an adjustable power amplifier, where one end of the adjustable power amplifier is connected to the other end of the LMX2594 chip, and the adjustable power amplifier is configured to receive the intervention signal and amplify the intervention signal; and
a thin-film lens microstrip line, where the thin-film lens microstrip line is connected to the other end of the adjustable power amplifier and configured to radiate the amplified intervention signal to a human brain, to complete one intervention stimulation on the human brain.

For example, the initial waveform (including a sine wave, a triangular wave, and a square wave) is selected through the LED display screen, and the time is set. The initial waveform is generated by the STM32 microcontroller unit. A serial peripheral interface (SPI) of the STM32 microcontroller unit is configured to be connected to the LMX2594 chip, and the initial waveform is input to the LMX2594 chip through the SPI. Meanwhile, phase-locked loop (PLL) parameters and frequency synthesizer settings may be configured. That is, configuration commands and data are sent to the LMX2594 chip through the SPI, to set the PLL parameters and relevant parameters of a frequency synthesizer. Then, an external reference clock signal is used as a reference clock for a PLL frequency divider of the LMX2594 chip. After that, the LMX2594 chip performs frequency division on the reference clock signal to reach a required frequency range. A PLL locks a phase of an output signal to a phase of a reference clock, thus maintaining stability and accuracy of the output signal. An N-times frequency divider of the PLL may be adjusted programmatically, such that the LMX2594 chip generates the required intervention signal based on the initial waveform, the processed human bioconductance data, and the temperature data. Then, the intervention signal is sent through a single channel of the LMX2594 chip, amplified by the adjustable power amplifier, and then radiated to the human brain through the thin-film lens microstrip line, to complete one intervention stimulation on the human brain.

In conclusion, the solution of the present disclosure has the following beneficial effects.

Firstly, conventional brain intervention has invasiveness and high energy, leading to a series of risks and complications. The present disclosure is a non-invasive intervention apparatus, and the apparatus reduces risks of existing brain intervention methods and decreases an incidence rate of complications and infections in invasive intervention.

Secondly, microwave brain intervention stimulation of the present disclosure may accurately locate a specific zone of a brain for treatment or research. Focused stimulation on brain zones may be implemented through an electrode design of the thin-film lens and the microstrip line.

Thirdly, tests show that the present disclosure achieves activation of relevant brain zones through power of 10 dBm. Meanwhile, with reference to a specific absorption rate, brain intervention using the solution of the present disclosure does not cause thermal effect damage. Thus, the solution of the present disclosure is lower in energy consumption and higher in safety.

Lastly, the present disclosure uses a printed gold-coated antenna. The apparatus is smaller in overall size, and has better biocompatibility, wide application scenes, and suitability for long-term wearing.

In one implementation, the apparatus further includes a pair of impedance electrodes; and an ADPD700 impedance analysis chip. One end of the ADPD700 impedance analysis chip is connected to the pair of impedance electrodes, and the other end of the ADPD700 impedance analysis chip is connected to the STM32 microcontroller unit. The ADPD700 impedance analysis chip is configured to collect and process the human bioconductance data through the pair of impedance electrodes, and send the processed human bioconductance data to the STM32 microcontroller unit. The human bioconductance data includes an impedance, a conductivity, and a permittivity of a human body.

For example, the ADPD700 impedance analysis chip of the apparatus of the present disclosure involves an algorithm fusing the impedance, the conductivity, and the permittivity of the human body. The algorithm fusing the impedance, the conductivity, and the permittivity of the human body is a method for analyzing and predicting bioconductance characteristics of the human body in different environments, and combines the impedance, the conductivity, and the permittivity of the human body, to improve accuracy and reliability of prediction. Specifically, the algorithm includes the following steps.

The human bioconductance data is collected through the pair of impedance electrodes and input into the ADPD700 impedance analysis chip. The human bioconductance data includes the impedance, the conductivity, and the permittivity of the human body.

The collected data is subjected to preprocessing operations, such as denoising, filtering, and normalization, to facilitate subsequent data analysis and modeling.

Valuable features are extracted from the preprocessed data. Specifically, the impedance, the conductivity, the permittivity, potential temporal and spatial correlations, and other features are extracted.

Based on the extracted features, a support vector machine (SVM) model is selected for training and prediction. Commonly used models include linear regression, a support vector machine, a neural network, and other models.

A validation set is used to evaluate the selected model, to determine performance, a generalization ability of the model for accuracy comparison.

The fused bioconductance characteristics of the human body are interpreted. According to the bioconductance characteristics of the human body at different body sites and under different parameters, data support is provided for electromagnetic characteristics of the human body under different environmental conditions.

Through the above steps, the algorithm fusing the impedance, the conductivity, and the permittivity of the human body may analyze and predict the bioconductance characteristics of the human body in different environments. The algorithm fusing the impedance, the conductivity, and the permittivity of the human body is used to determine a depth and size of a nucleus that needs intervention, calculate intervention resolution, and determine frequency and power. Thus, problems of inaccurate targeting and poor effects in existing transcranial alternating current stimulation and transcranial direct current stimulation technologies are solved.

Further, corresponding frequencies in an industrial, scientific and medical (ISM) band and an ultra-wideband band (UWB) band are selected for intervention, to solve a problem of personalized intracranial nucleus intervention, i.e., a problem of an intervention depth. Steps for predicting the intervention depth are as follows:

When passing through human tissue, electromagnetic waves may generate loss. The human body is essentially an inhomogeneous medium, so an attenuation magnitude depends on electromagnetic wave frequency and power, a tissue water content, and a target nucleus depth. A formula for energy loss of the electromagnetic waves in biological tissue is as follows:

$$P L ( d ) = 20 {log}_{10} ( \frac{4 π d f}{c} ) + K$$

In the formula, PL(d) denotes total energy loss, c denotes a speed of light, f denotes the intervention signal, K denotes a path loss constant, and is mainly determined by conductivities and permittivities of the tissue at different frequencies, and d denotes the intervention depth.

Because the human body is a lossy medium and frequency-related dielectric parameters of the human body have real parts, a wavelength of the electromagnetic waves may become shorter after penetrating into the human body. This also causes attenuation of the electromagnetic waves in the human body to occur with frequency, and an electric field intensity in the human tissue may be expressed as a function for propagating d along an x axis as follows:

$${E}_{z} = {E}_{z 0} {e}^{j ( ω t - kd )}$$

In the function, E_{z0} denotes an electric field intensity at an air bath-human body interface, and e^{j(ωt-kd)} denotes a loss factor of the electromagnetic waves in a propagation direction.

An adaptive positioning frequency may be obtained by combining optimal solutions of the above two equations. That is, finally, the above features are fused, classification is performed according to the fused features, and an appropriate carrier frequency is selected.

It should be understood that intracranial nuclei refers to a collection of neurons located inside the brain, and play an important regulatory role in a nervous system. The nuclei are generally composed of similar types of neurons and are closely connected functionally to form specific neural circuits. The intracranial nuclei play an important role in human movement, emotion, cognition, sensation, and other aspects.

In another implementation, the apparatus further includes a temperature probe; and an M601 body temperature monitoring chip. One end of the M601 body temperature monitoring chip is connected to the temperature probe, and the other end of the M601 body temperature monitoring chip is connected to the STM32 microcontroller unit. The M601 body temperature monitoring chip is configured to monitor changes in surface temperature of a scalp through the temperature probe, generate the temperature data, and send the temperature data to the STM32 microcontroller unit.

For example, the M601 body temperature monitoring chip of the apparatus of the present disclosure involves surface temperature safety evaluation. Specific steps are as follows.

Data is collected through the temperature probe attached to the scalp, input into an analog-to-digital converter (ADC) of the M601 body temperature monitoring chip, and then forwarded to the microcontroller unit (MCU). Collection accuracy is ±0.1° C., and collection frequency is 1 time/S.

Normalization and standardization are performed on the temperature data, power data, and current data, to prevent difficulty in training convergence. Specifically, a feature mean is subtracted from the data, a result is divided by a standard deviation, and finally mean and standard deviation transformation is performed.

A keras deep learning framework is used to build a 3-layer artificial neural network (ANN) model for training a small-sample data set. A data dimension is based on stimulation duration of 20 minutes, and data includes temperatures, power, and currents. An input data set dimension is (1200, 3), and then K-fold cross-validation is used.

Model training and evaluation are performed, with indicators including a mean squared error (MSE) and a mean absolute error (MAE).

After a plurality of iterations, a minimum MAE value is found, and corresponds to an abnormal temperature rise and a specific absorption rate (SAR) threshold.

A surface temperature and an SAR parameter of a target zone are evaluated to ensure safety of stimulation.

In another implementation, the thin-film lens microstrip line includes a thin-film lens and a microstrip line. The thin-film lens is configured to refract the intervention signal, causing the intervention signal to be focused on the microstrip line, to implement intervention stimulation on a specific zone of the human brain.

For example, in the embodiment of the present disclosure, the thin-film lens microstrip line is designed as an efficient microwave transmission structure. A possible design is described as follows.

A structure of the thin-film lens microstrip line is as follows.

A basic structure of a front end of the thin-film lens microstrip line is shown in FIG. 2. The thin-film lens microstrip line is mainly composed of the thin-film lens, for example, a lens composed of a dielectric layer having an appropriate refractive index, and the microstrip line.

A function of the thin-film lens microstrip line is as follows.

A main function of the thin-film lens microstrip line is to focus a microwave signal on a zone that needs intervention stimulation. Specifically, after the microwave signal is transmitted to the front end of the thin-film lens microstrip line, through a refraction effect of the lens, the microwave signal is focused on the microstrip line to form a zone having a high power density, such that intervention stimulation on the specific zone of the brain is implemented.

Design requirements of the thin-film lens microstrip line are as follows.

To achieve efficient microwave transmission and focusing, the thin-film lens microstrip line needs to satisfy the following requirements: The lens should have an appropriate refractive index to ensure that the intervention signal, i.e., the microwave signal, may pass through the lens smoothly and be focused on the microstrip line. The design of the microstrip line should ensure that the microwave signal has no loss during transmission and has a sufficient bandwidth, such that the microwave signal may be fully focused at the front end of the microstrip line. A distance between the thin-film lens and the microstrip line should be appropriate to ensure that the microwave signal may maintain a focused state during transmission. The front end of the thin-film lens microstrip line should have desirable electromagnetic compatibility to ensure that the microwave signal may work normally in a complex electromagnetic environment.

By satisfying the above design requirements, the thin-film lens microstrip line may achieve efficient microwave transmission and focusing functions of an apparatus for non-invasive brain intervention with radio frequency microwaves, and solve a problem of electromagnetic wave dispersion in brain zones during radiation.

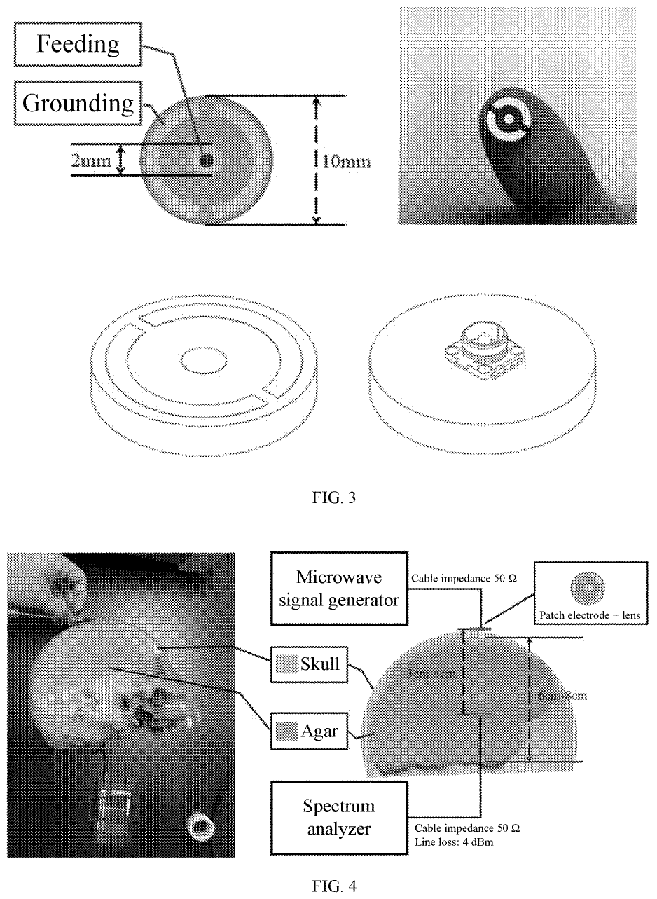

Preferably, with reference to FIG. 3 and FIG. 4, the present disclosure further provides a sensor at a front end of a microstrip line. The sensor uses a double-sided printing structure. An IPEX socket is mounted on a reverse side of the structure. In FIG. 3, a light gray part is a dielectric substrate made of alumina ceramic, with a stable dimension, a relative permittivity between 5 and 10, excellent bending resistance, flame retardancy and insulation properties, and an electrode part made of gold. In one embodiment, specific surface structure parameters are shown in FIG. 3, and parameters of a substrate of a single patch unit are 10 mm×10 mm×1.6 mm.

A double-sided printing microstrip line structure having a plurality of working frequencies provided by the present disclosure is divided into two parts in terms of feeding positions. A first part is a central feeding position part. A second part includes arc-shaped grounding parts at two sides. The structure has desirable notch characteristics for signals mainly in a frequency band of 2 GHz to 12 GHz.

Generally, for a central feed antenna, if multi-pole antennas are too close to each other, mutual interference may occur, mainly due to conduction of interference clutter by currents on a printed copper clad. However, in an antenna board of the present disclosure, a current path from a feed point to a polarization point changes according to a branch direction. Meanwhile, different dipole directions and different structures enable desirable adjustment of phases of antenna surfaces at three polarization positions, thus reducing interference between current conduction. Grounding positions are designed on two sides of the substrate, and the design not only effectively eliminates interference caused by a grounding effect, but also ensures vertically downward radiation emitted by the antenna to a certain extent. Thus, directionality of conduction in front and rear directions of the antenna is enhanced. In addition, it is ensured that the antennas of three branches have desirable isolation.

In terms of materials, the front-end microstrip line uses an alumina ceramic plate as the substrate. The microstrip line designed in the present disclosure is attached to the scalp, and the alumina ceramic plate has better applications in aerospace, military industry, and medical fields.

Firstly, the alumina ceramic substrate has higher hardness and is not easy to damage during repeated use.

Secondly, the alumina ceramic substrate has better biocompatibility, and is not likely to breed bacteria or cause skin allergies during long-term use.

In addition, the alumina ceramic substrate has good insulation performance, thus failing to easily produce other interference and maintaining stability of intervention energy.

In terms of structures, a microstrip antenna patch branch structure designed in the present disclosure is simple. According to the branch structure, Balun triangle parameters forming a trapezoid at two sides of each asymmetric dipole and a diameter of a circular part may be adjusted to easily design a microstrip line antenna patch adapted to frequencies of other communication apparatuses. Meanwhile, the antenna designed in the present disclosure has a small size, and is suitable for mounting on a wheelchair device, such that miniaturization of the apparatus is made possible.

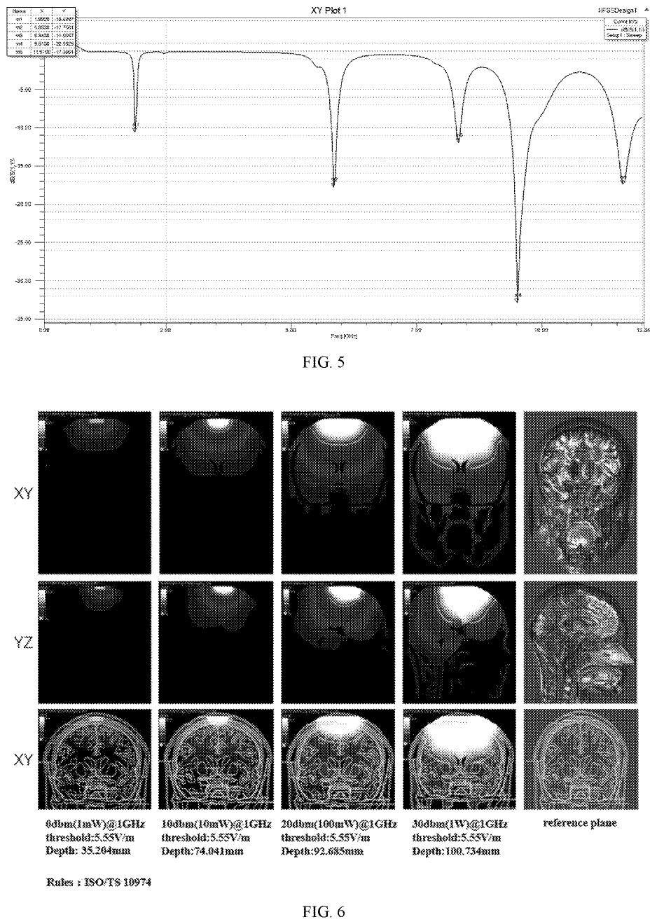

After simulation, as shown in FIG. 5, it may be seen that the structure has better notch characteristics at a plurality of working frequencies of ISM and UWB (2 GHz, 6 GHz, 8.5 GHz, 9.5 GHz, and 11.5 GHz). From simulation results in FIG. 5, isolation between all working frequencies is desirable, such that possible inter-frequency interference is reduced.

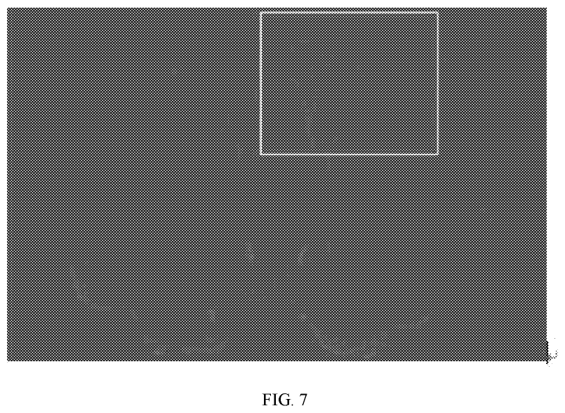

With reference to FIG. 6, a technology in the present disclosure performs simulation through sim4life bioelectromagnetic simulation software. With pulsed electromagnetic waves of 1 GHz to 4 GHz (with an electric field intensity threshold of 5.55 V/m) without a specific target spot as an example, microwave intervention characteristics are studied, and simulation verification is performed on XY, YZ, and XZ planes separately. The solution of the present disclosure has the following two characteristics.

As power continues to increase, an intervention depth becomes greater, and accuracy gradually decreases.

At identical power, the intervention depth may change with change of frequency.

Meanwhile, to test effectiveness, a method of pouring agar into a real skull is used to measure attenuation of the microwave signal. A transmission end uses a microwave signal generator, and a reception end uses a frequency spectrograph, as shown in FIG. 7. Transmission power is 10 dBm, and data collection is performed at 2 cm, 5 cm, and 8 cm inside the skull. For each set, 2,000 pieces of reception power data are measured and fitted.

TABLE 1
Fitting results

| | 2 cm | | 5 cm | | 8 cm | |
|---|---|---|---|---|---|---|
| | Mean ± | | Mean ± | | Mean ± | |
| | standard | | standard | | standard | |
| | deviation | Optimal | deviation | Optimal | deviation | Optimal |
| Item | (dBm) | fit | (dBm) | fit | (dBm) | fit |
| Test group | 9.1 ± 0.4 | G | 7.6 ± 0.8 | W | 5.3 ± 1.5 | N |

| | |
|---|---|
| Fading model | G: Gamma N: Nakagami W: Weibull |

As shown in Table 1, the data in Table 1 indicates that as an intervention distance increases, relevant data is fitted to conform to Gamma distribution, Nakagami distribution, and Weibull distribution, respectively, showing that energy received by target brain region nuclei decreases, that is, a received power value is decreased. Meanwhile, it may be seen that in addition to mean path loss and multi-medium loss in an energy path, there is also multipath dispersion in small-scale fading. Relevant animal experiments show that nerve cells may still be activated.

In addition, the present disclosure performs simulation on safety of electromagnetic waves. At present, evaluation of safety of electromagnetic wave radiation to the human body is mainly based on a specific absorption rate (SAR) value. The specific absorption rate is defined as electromagnetic power absorbed per unit mass of human tissue (W/Kg), and the formula is defined as follows:

$$SAR = \frac{σ}{ρ} {E}^{2}$$

In the formula, ρ denotes a mass density of the tissue, and E denotes a root mean square of an electric field intensity.

The following data is obtained through sim4life simulation. After simulation calculation is completed, simulation data is recorded as shown in Table 2.

TABLE 2
Simulation data

| Human organ | SAR value (W/kg) |
|---|---|
| Coupling position | Calvarium |
| Brain | 5.53 × 10⁻³ |

By analyzing the data in Table 2, it may be seen that when an intervention electrode is placed at a position of a calvarium, a maximum SAR value is 5.53×10⁻³ W/kg, and an organ is a brain (including gray matter, white matter, etc.). The maximum SAR value appearing at the position is much less than an SAR safety limit of 2 W/kg. Thus, a non-invasive microwave brain intervention technology is a new intervention method with low radiation and high safety.

The present disclosure collects an impedance situation of the human body and corresponding target brain zone nuclei, and analyzes an optimal electrode placement position, an intervention signal and power according to electromagnetic wave frequency attenuation characteristics, to implement non-invasive intervention on the target nuclei. In addition, the present disclosure combines adaptive selection to locate an optimal working frequency. Through electromagnetic detection around the human body, an intervention signal having less interference is adaptively selected, and a possibility of reducing intervention effectiveness due to large skin components is reduced from the source. Through the proposed design of front-end units of the thin-film lens microstrip line, the units may work at a plurality of frequency bands, a multi-unit matrix composed of such units is designed, and a composite intervention array is formed by multi-frequency monopoles. Thus, simultaneous multi-target-spot intervention is effectively increased, and brain zone intervention linkage is made possible. In terms of safety, temperature changes on a scalp surface are monitored in real time to strictly prevent possible thermal effect damage.

Thus, the apparatus for non-invasive brain intervention with radio frequency microwaves according to the present disclosure has the following advantages.

Firstly, the apparatus integrates an advanced brain positioning technology with a microwave thin-film lens technology, such that precise positioning of a brain stimulation zone may be implemented, thus improving a treatment effect.

Secondly, there are mainly two biological effects of an electromagnetic field: one of the effects is a stimulation effect on a nervous system caused by induced electric and magnetic fields in human tissue; and the other is a thermal effect caused by absorption of electromagnetic energy by human tissue. The present disclosure mainly uses the first effect. Thus, microwave radiation energy is much lower than a safety limit of an international specific absorption rate.

Lastly, microwave energy does not directly generate currents, and forms a composite electromagnetic field and has good penetrability. Propagation characteristics of microwaves in the human body are mainly related to frequencies, and the frequencies affect a skin depth. Meanwhile, dielectric properties and conductivities at different frequencies are varied, so electrodes may form stimulation without completely making contact with skin.

Thus, specific embodiments of the present disclosure are described. Other embodiments fall within the scope of the appended claims. In some cases, actions recited in the claims may be performed in a different order and still achieve a desired result. In addition, processes depicted in the drawings do not necessarily require a specific order or a continuous order shown to achieve the desired result. In some implementations, multitasking and parallel processing may be advantageous.

It should be noted that all directional indications (such as upper, lower, left, right, and rear) in the embodiments of the present disclosure are only used to describe a relative positional relationship, a motion situation, etc. among components in a certain posture (as shown in the drawings). If the specific posture changes, the directional indications will change accordingly.

The terms such as “first” and “second” in the description of the present disclosure are merely used to conveniently describe different components or names, and cannot be construed as indicating or implying their ordinal relationships and relative importance or implicitly specifying a quantity of indicated technical features. Thus, a feature defined with “first” and “second” may explicitly or implicitly include at least one of the features.

Unless otherwise defined, all technical and scientific terms used herein have identical meanings to those commonly understood by those skilled in the technical field of the present disclosure. The terms used in the description of the present disclosure herein are just for the purpose of describing specific embodiments, and are not intended to limit the present disclosure.

It should be noted that although the specific embodiments of the present disclosure are described in detail with reference to the accompanying drawings, the specific embodiments should not be construed as limiting the protection scope of the present disclosure. Various modifications and variations that may be made by those skilled in the art without creative efforts within the scope described in the claims should still fall within the protection scope of the present disclosure.

Examples of the embodiments of the present disclosure are intended to briefly illustrate technical features of the embodiments of the present disclosure, and those skilled in the art may intuitively understand the technical features of the embodiments of the present disclosure. The technical features do not serve as an improper limitation to the embodiments of the present disclosure.

Finally, it should be noted that the above embodiments are only used to illustrate the technical solution of the present disclosure, instead of limiting the technical solution. Although the present disclosure is described in detail with reference to the above embodiments, those of ordinary skill in the art should understand that the technical solution described in the above embodiments may still be modified, or some technical features may still be replaced with equivalents. However, these modifications or replacements do not make the essence of the corresponding technical solution deviate from the spirit and scope of the technical solution of the embodiments of the present disclosure.

What is claimed is:

1. An apparatus for non-invasive brain intervention with radio frequency microwaves, comprising:
a light emitting diode (LED) display screen, wherein the LED display screen is configured to select a to-be-generated initial waveform and set time;
a microcontroller unit, wherein one end of the microcontroller unit is connected to the LED display screen, and the microcontroller unit is configured to generate the initial waveform;
a chip, wherein one end of the chip is connected to the other end of the microcontroller unit, and the chip is configured to generate and send an intervention signal according to the initial waveform, processed human bioconductance data, and temperature data;
an adjustable power amplifier, wherein one end of the adjustable power amplifier is connected to the other end of the chip, and the adjustable power amplifier is configured to receive the intervention signal and amplify the intervention signal; and
a thin-film lens microstrip line, wherein the thin-film lens microstrip line is connected to the other end of the adjustable power amplifier and configured to radiate the amplified intervention signal to a human brain, to complete one intervention stimulation on the human brain.

2. The apparatus according to claim 1, further comprising:
a pair of impedance electrodes; and
an impedance analysis chip, wherein
one end of the impedance analysis chip is connected to the pair of impedance electrodes, the other end of the impedance analysis chip is connected to the microcontroller unit, the impedance analysis chip is configured to collect and process the human bioconductance data through the pair of impedance electrodes, and send the processed human bioconductance data to the microcontroller unit, and the human bioconductance data comprises an impedance, a conductivity, and a permittivity of a human body.

3. The apparatus according to claim 2, further comprising:
a temperature probe; and
a body temperature monitoring chip, wherein
one end of the body temperature monitoring chip is connected to the temperature probe, the other end of the body temperature monitoring chip is connected to the microcontroller unit, and the body temperature monitoring chip is configured to monitor changes in surface temperature of a scalp through the temperature probe, generate the temperature data, and send the temperature data to the microcontroller unit.

4. The apparatus according to claim 3, wherein the thin-film lens microstrip line comprises a thin-film lens and a microstrip line, and the thin-film lens is configured to refract the intervention signal, causing the intervention signal to be focused on the microstrip line, to implement intervention stimulation on a specific zone of the human brain.
